(19) United States
(12) Patent Application Publication
(10) Pub. No.: US 2005/0090449 A1
(43) Pub. Date: Apr. 28, 2005

(54) Novel statine derivatives for the treatment of Alzheimer's disease

(75) Inventors: Klaus Fuchs, Schemmerhofen (DE); Stefan Peters, Biberach (DE); Comelia Dorner-Ciossek, Ravensburg (DE); Marcus Kostka, Biberach (DE); Sandra Handschuh, Warthausen (DE); Christian Haass, Icking (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/840,037

(22) Filed: May 6, 2004

Related U.S. Application Data
(60) Provisional application No. 60/497,613, filed on Aug. 25, 2003.

(30) Foreign Application Priority Data
May 13, 2003 (EP) 03010662.9

(51) Int. Cl. A61K 38/08; A61K 38/06; A61K 38/05; A61K 38/04; C07K 7/06; C07K 5/06

(52) U.S. Cl. 514/17; 514/18; 514/19; 530/329; 530/330; 530/331

(57) ABSTRACT

The invention relates to a compound of the formula 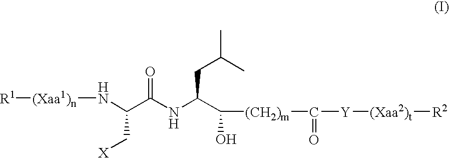
wherein R₁, R², X, Y, n, t and m are defined as in the specification and claims and to its use for treating or preventing Alzheimer's disease and other similar diseases.

### RELATED APLLICATIONS

This application claims benefit of U.S. Ser. No. 60/497,613, dated Aug. 25, 2003, and claims priority to European Application No. 03010662.9, dated May 13, 2003, each of which is incorporated by reference in its entirety.

### BACKGROUND OF THE INVENTION

1. Technical Field

The invention relates to novel statine derivatives and to their use for treating or preventing Alzheimer's disease and other similar diseases.

2. Background Information

Alzheimer's disease (AD) is a progressive degenerative disease of the brain primarily associated with aging. Clinical presentation of AD is characterized by loss of memory, cognition, reasoning, judgement, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD.

Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i. e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage site. The beta-secretase enzyme has been disclosed using varied nomenclature, including BACE, Asp2, am Memapsin2. See, for example, Sindha et. al., 1999, Nature 402 : 537-554 and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6: 487-498. Release of A beta from neuronal cells grown in culture and the presence of A beta in cerebrospinal fluid (CSF) of both normal individuals and AD patients has been demonstrated. See, for example, Seubert et al., 1992, Nature 359: 325-327.

It has been proposed that A beta peptide accumulates as a result of APP processing by beta-secretase, thus inhibition of this enzyme's activity is desirable for the treatment of AD, see for example Vassar, R. 2002, Adv. Drug Deliv. Rev. 54, 1589-1602 In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that overexpress APP, the progeny show reduced amounts of A beta in brain extracts as compared with control animals (Luo et. al., 2001 Nature Neuroscience 4: 231-232). This evidence further supports the proposal that inhibition of beta-secretase activity and reduction of A beta in the brain provides a therapeutic method for the treatment of AD and other beta amyloid disorders.

The International patent application WO00/47618 identifies the beta-secretase enzyme and methods of its use. This publication also discloses oligopeptide inhibitors that bind the enzyme's active site and are useful in affinity column purification of the enzyme. In addition, WO00/77030 discloses tetrapeptide inhibitors of beta-secretase activity that are based on a statine molecule.

Various pharmaceutical agents have been proposed for the treatment of Alzheimer's disease but without any real success. U.S. Pat. No. 5,175,281 discloses aminosteroids as being useful for treating Alzheimer's disease. U.S. Pat. No. 5,502,187 discloses bicyclic heterocyclic amines as being useful for treating Alzheimer's disease.

EP 652 009 A1 discloses inhibitors of aspartyl protease which inhibit beta amyloid peptide production in cell culture and in vivo. The compounds which inhibit intracellular beta-amyloid peptide production are useful in treating Alzheimer's disease.

WO00/69262 discloses a new beta-secretase and its use in assays to screen for potential drug candidates against Alzheimer's disease.

WO01/00663 discloses memapsin 2 (human beta-secretase) as well as catalytically active recombinant enzyme. In addition, a method of identifying inhibitors of memapsin 2, as well as two inhibitors are disclosed. Both inhibitors that are disclosed are peptides.

WO01/00665 discloses inhibitors of memapsin 2 that are useful in treating Alzheimer's disease.

At present there are no effective treatments for halting, preventing, or reversing the progression of Alzheimer's disease. Therefore, there is an urgent need for pharmaceutical agents with sufficient plasma and/or brain stability capable of slowing the progression of Alzheimer's disease and/or preventing it in the first place.

Compounds that are effective inhibitors of beta-secretase, that inhibit beta secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

### BRIEF SUMMARY OF THE INVENTION

Surprisingly, it has been found that statine derivatives, wherein a norvaline, a cycloalkylalanin or a (R)-methylcystein group is attached to the 4-amino group of the statine moiety, show superior inhibition of beta secretase-mediated cleavage of APP and sufficient plasma stability. Surprisingly, substitution of asparagine in P2 position by small aliphatic amino acids were found active and improved physicochemical properties.

Thus the invention relates to a compound of the formula 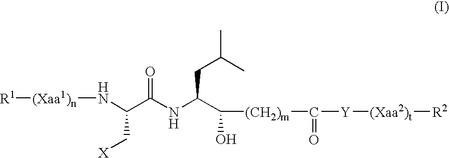
wherein
R¹ represents a hydrogen atom or a group selected from the formulae (A) and (B)
   (A) R³—CO—(CH₂)ₛ—CO—,
      in which
      R³ represents R⁴-Z¹ with Z¹ being O or NR⁵, R⁴ and R⁵ being each
      independently hydrogen or C₁₋₆ alkyl, and
      s is an integer from 1 to 4;
   (B) R⁶—CO—
      in which
      R⁶ represents a C₁₋₆ alkyl group, a C₁₋₆ haloalkyl group or a phenyl group being optionally substituted by one or more substituents selected from the group consisting of halogen, C₁₋₆ alkyl, C₁₋₆ alkoxy, C₁₋₆ haloalkyl, C₁₋₆ haloalkoxy, amino, C₁₋₆ alkylamino, di-(C₁₋₆ alkyl)-amino, C₁₋₆ alkanoylamino, C₁₋₆ alkoxycarbonyl, formyl, carboxy, hydroxy, SO₃H, cyano and nitro;
Xaa¹ each independently represent an amino acid or the N-alkylated derivative thereof, at least one of which being N-terminally linked to R¹;
n is 0 or an integer from 1 to 3;
Y represents a single bond, or if t is 0, a spacer group selected from —O— and —NH—;
R² represents a hydroxy group or a group of formula (C)
(C) -Z²-R⁷
   in which
   Z² represents O or NR⁸,
   R⁷ represents
   (a) a C₁₋₆ alkyl group being optionally substituted by one or more substituents selected from the group consisting of halogen, C₃₋₈-cycloalkyl, phenyl, C₁₋₆ alkoxy, C₁₋₆ haloalkoxy, amino, C₁₋₆ alkylamino, di-(C₁₋₆ alkyl)-amino, C₁₋₆ alkoxycarbonyl, formyl, carboxy, hydroxy, cyano and nitro, or
   (b) a phenyl group being optionally substituted by one or more substituents selected from the group consisting of halogen, C₁₋₆ alkyl, C₁₋₆ alkoxy, C₁₋₆ haloalkyl, C₁₋₆ haloalkoxy, amino, C₁₋₆ alkylamino, di-(C₁₋₆ alkyl)-amino, C₁₋₆ alkoxycarbonyl, formyl, carboxy, hydroxy, cyano and nitro,
   R⁸ represents a hydrogen atom or C₁₋₆ alkyl group;
Xaa² each independently represent an amino acid or the N-alkylated derivative thereof, in which the amino group of the N-terminally amino acid may have been replaced by
Y, and one of which being C-terminally linked to R²
t is 0 or an integer from 1 to 3;
X is selected from ethyl, thiomethyl and C₃-C₈-cycloalkyl; and
m is 1 or 2.
   or a pharmaceutically acceptable salt or solvate thereof.

Furthermore, the invention relates to a pharmaceutical composition comprising a compound of formula I or a pharmaceutically acceptable salt or solvate thereof and a pharmaceutically acceptable carrier or diluent.

Another aspect of the present invention is the use of a compound of formula I or a pharmaceutically acceptable salt or solvate thereof in the manufacture of a medicamentation for use in treating a patient who has, or in preventing a patient from getting, a disease or condition selected from Alzheimer's disease, Down's syndrome, MCI (“Mild Cognitive Impairment”), Hereditary Cerebral Hemorrhage with Amyloidosis of the Dutch-Type, Cerebral Amyloid Angiopathy, Traumatic Brain Injury, Stroke, Dementia, Parkinson's Disease and Parkinson's Syndrome, or central or peripheral amyloid diseases.

Furthermore the invention relates to a method for inhibiting β-secretase activity, comprising exposing said β-secretase to an effective inhibitory amount of a compound of formula I.

The present invention provides compounds, compositions, kits, and methods for inhibiting beta-secretase-mediated cleavage of amyloid precursor protein (APP).

More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds of the invention possess beta-secretase inhibitory activity.

The inhibitory activities of the compounds of the invention are readily demonstrated, for example, using one or more of the assays described herein or known in the art.

### DETAILED DESCRIPTION OF THE INVENTION

The present invention is the substituted amines (I) that are useful in treating and preventing Alzheimer's disease.

The term alkyl groups (including those which are part of other groups, especially alkoxy), unless otherwise stated, denotes branched and unbranched alkyl groups with 1 to 6 carbon atoms, preferably 1 to 4 carbon atoms, most preferably 1 to 3 carbon atoms, especially 1 or 2 carbon atoms. Examples are: methyl, ethyl, propyl, butyl, pentyl, hexyl, etc. Unless otherwise stated, the above terms propyl, butyl, pentyl or hexyl also include all the possible isomeric forms. For example, the term propyl also includes the two isomeric groups n-propyl and iso-propyl, the term butyl includes n-butyl, iso-butyl, sec. butyl and tert.-butyl, the term pentyl includes iso-pentyl, neopentyl, etc. In some cases common abbreviations are also used to denote the above mentioned alkyl groups, such as Me for methyl, Et for ethyl etc.

The term haloalkyl groups (including those which are part of other groups, especially haloalkoxy), unless otherwise stated, denotes branched and unbranched haloalkyl groups with 1 to 6 carbon atoms, preferably 1 to 4 carbon atoms, especially 1 to 3 carbon atoms, which are substituted by at least one halogen atom, particularly fluorine atom. Fluorinated groups of the formula
—(CH₂)ₚ—(CF₂)_{q}—y
wherein
p denotes 0 or an integer from 1 to 3,
q denotes an integer from 1 to 3, and
Y denotes hydrogen or fluorine, are preferred.

Examples include: trifluoromethyl, trifluoromethoxy, difluoromethoxy, perfluoroethyl, perfluoropropyl, 2,2,2-trifluoroethyl, 2,2,2-trifluoroethoxy, 1,1,1-trifluoroprop-2-yl, etc.

The term halogen generally denotes fluorine, chlorine, bromine or iodine.

The term cycloalkyl groups (including those which are part of other groups, especially cycloalkoxy), unless otherwise stated, denotes cyclic alkyl groups with 3 to 8 carbon atoms, preferably 3 to 6 carbon atoms, most preferably 3, 5 or 6 carbon atoms, especially 3 carbon atoms. Examples are: cyclopropyl, cyclobutyl, cyclopentyl, cyclohexyl, etc. Most preferred is cyclopropyl.

Preferred are the compounds of formula (I), wherein
Xaa¹ each independently is selected from the group of amino acids consisting of Leu (leucine), Ile (isoleucine), Nva (norvaline, 2-amino-pentanoic acid)), Abu (2-amino-butyric acid), Glu (glutamic acid), Tle (tert.-leucine, 2-amino-3,3-dimethyl-butyric acid), Phg (phenylglycine), Val (valine), allo-Ile ((2S,3S)-2-amino-3-metyl-pentanoic acid), Cpa (beta-cyclopropyl-alanine), Met (methionine), Thr (threonine), Chg (cyclohexylglycine), S-methylcysteine, D-Leu, Nip (nipecotic acid, piperidine-3-carboxylic acid), CBA (cyanobutyric acid) and allyl-glycine, in particular Leu, Ile, Cpa and Glu
n is 1 or 2; and/or wherein
Xaa² each independently is selected from the group of amino acids consisting of Val, Ala, Leu, Ile, Nva, Abu, Cha, Tle, Phg, Glu, Nle, Phe (phenylalanine), His (histidine), Ser (serine), Cpa and Asp, in particular Nva, Val, Cpa and Ala.
s is 1 or 2.

Furthermore preferred are those compounds of formula (I), wherein
R¹ represents a hydrogen atom or a group selected from the formulae (A) and (B)
   (A) R³—CO—(CH₂)ₛ—CO—,
      in which
      R³ represents R⁴—O, R⁴ being each independently hydrogen or C₁₋₃ alkyl and
      s is 1 or 2;
   (B) R⁶—CO—
      in which
      R⁶ represents a C₁₋₃ alkyl group, a C₁₋₃ haloalkyl group or a phenyl group being substituted by one or two substituents selected from the group consisting of halogen, C₁₋₃ alkyl, C₁₋₃ alkoxy, C₁₋₃ haloalkyl, C₁₋₃ haloalkoxy, amino, C₁₋₃ alkylamino, di-(C₁₋₃ alkyl)-amino, C₁₋₃ alkanoylamino, C₁₋₃ alkoxycarbonyl, formyl, carboxy, hydroxy, SO₃H, cyano and nitro;
Xaa¹ each independently represent an amino acid, at least one of which being N-terminally linked to R¹;
n is 1 or 2;
Y represents a spacer group selected from —O— and —NH—;
R² represents a hydroxy group or a group of formula (C)
(C) -Z²-R⁷
   in which
   Z² is NR⁸,
   R⁷ represents
   (a) a C₁₋₃ alkyl group, or
   (b) a phenyl group being optionally substituted by one or more substituents selected from the group consisting of halogen, C₁₋₃alkyl, C₃ alkoxy, C₁₋₃ haloalkyl, C₁₋₃ haloalkoxy, amino, C₁₋₃ alkylamino, di-(C₁₋₃ alkyl)-amino, C₁₋₃ alkoxycarbonyl, formyl, carboxy, hydroxy, SO₃H, cyano and nitro,
   R⁸ represents a hydrogen atom;
Xaa² each independently represent an amino acid or the N-alkylated derivative thereof, in which the amino group of the N-terminally amino acid may have been replaced by Y, and one of which being C-terminally linked to R²;
t is an integer from 1 to 3;
X is selected from ethyl, thiomethyl and C₃-C₆-cyclomethyl; and
m is 1 or 2.
   or a pharmaceutically acceptable salt or solvate thereof.

Another group of preferred compounds of formula (I) are those, wherein
R¹ represents a hydrogen atom or a group selected from the formulae (A) and (B)
   (A) R³—CO—(CH₂)ₛ—CO—,
      in which s has the meaning given, and
      R³ represents R⁴—O, and R⁴ being each independently hydrogen or methyl;
   (B) R⁶—CO—
      in which
      R⁶ represents a phenyl group being substituted by one substituent selected from the group consisting of acetylamino, hydroxy, SO₃H, and carboxy;
Xaa¹ each independently represent an amino acid, at least one of which being N-terminally linked to R¹;
n is 1 or 2;
Y represents a single bond,
R² represents a hydroxy group or a group of formula (C)
(C) -Z²-R⁷
   in which
   Z² is NR⁸,
   R⁷ represents a C₁₋₃ alkyl group,
   R⁸ represents a hydrogen atom;
Xaa² each independently represent an amino acid, in which the amino group of the N-terminally amino acid may have been replaced by Y, and one of which being C-terminally linked to R²;
t is 1 or 2;
X is selected from ethyl, thiomethyl and C₃-C₆-cyclomethyl; and
m is 1 or 2.
   or a pharmaceutically acceptable salt or solvate thereof.

Particularly preferred are the compounds of formula (I), wherein m is 1.

Furthermore preferred are those compounds of formula (I), wherein
(a) n is 1; and R¹ represents R³—CO—(CH₂)ₛ—CO— (A) or R⁶—CO— (B), in which R³, R⁶ and s have the meaning given hereinbefore; or
(b) n is 2, the N-terminal group Xaa¹, which is attached to R¹, represents Glu, and R¹ represents a hydroxy group.

Most preferred are the compounds of formulae (IA) to (ID): 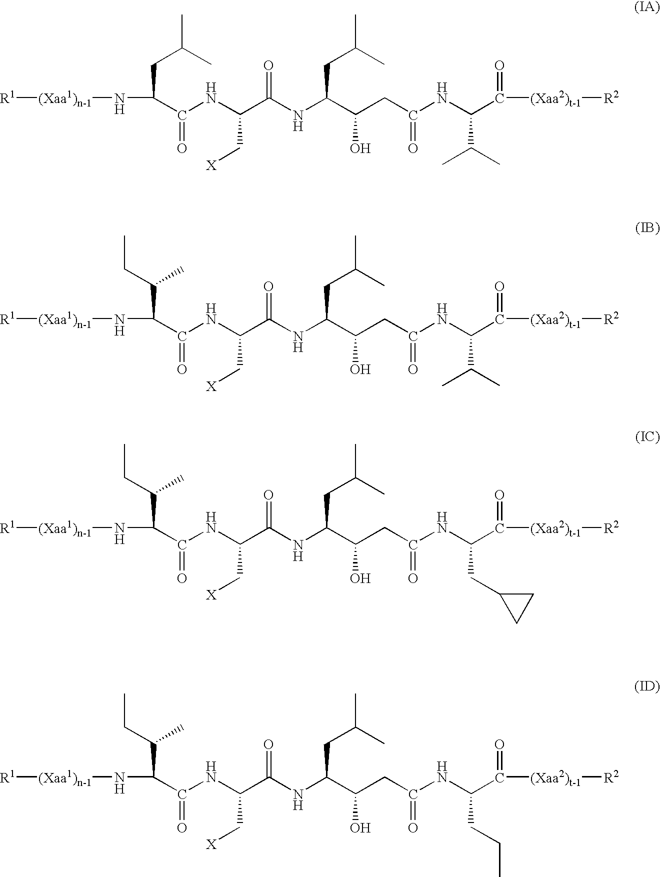
in which R¹, R², Xaa¹, Xaa², n and t are as defined hereinbefore, and X represents ethyl or cyclopropyl; or a pharmaceutically acceptable salt or solvate thereof.

The anti-Alzheimer's substituted amines (I) and (IA) through (ID) are made by methods well known to those skilled in the art from starting compounds known to those skilled in the art. The process chemistry is well known to those skilled in the art. The following reaction schemes illustrate the peptide synthesis of the statine derivatives according to the present invention.

One skilled in the art will appreciate that these are all well known reactions in organic chemistry (Houben-Weyl—Methods of Organic Chemistry, Vol E22, Synthesis of Peptides and Peptidomimetics, M. Goodman, A. Felix, L. Moroder, C. Toniolo Eds., Georg Thieme Verlag Stuttgart, New York). A chemist skilled in the art, knowing the chemical structure of the biologically active substituted amine end product (I) of the invention would be able to prepare them by known methods from known starting materials without any additional information. The explanation below therefore is not necessary but is deemed helpful to those skilled in the art who desire to make the compounds of the present invention.

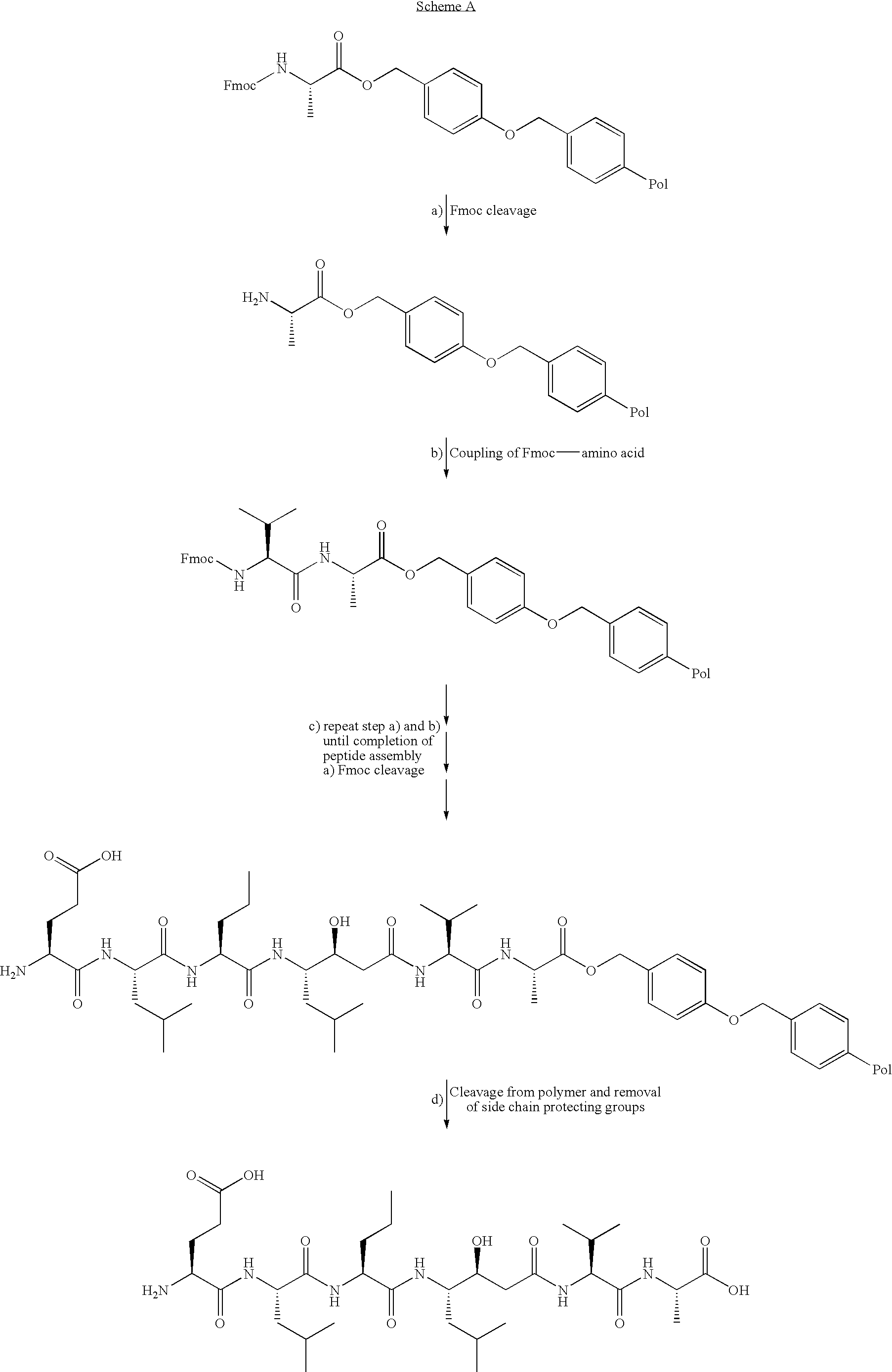

As illustrated in scheme A the synthesis of peptides bearing the free carboxy-terminus can be performed by standard peptide chemistry applying the Fmoc/tBu-protection. The first amino acid (Fmoc-alanine) has been esterified with the Wang-resin. The Fmoc-Ala-Wang resin is commercially available. After deprotection of the Fmoc-group (step a) the next amino acid (Fmoc-valine) is coupled with a suitable peptide coupling reagent such as TBTU/HOBt (step b). The peptide assembly is reapeated applying step a) and b) and using the respective amino acids Fmoc-statine, Fmoc-Nva, Fmoc-Leu and Fmoc-Glu(tBu) until completion of the peptide chain. After removal of the terminal Fmoc-group the peptide is cleaved from the polymer with trifluoroacetic acid with concurrent removal of the tBu-side chain protecting group of the glutamic acid residue. The crude peptide can be purified by precipitation from diethyl ether and by reversed phase HPLC.

The synthesis protocol allows the incorporation of different amino acid residues in the position Xaa1 and Xaa2 of formula (I) and the variation of the peptide length n, s and t in formula (I) as well. The substituent X of formula (I) can also be varied by incorporation of a suitable amino acid.

A slightly modified solid-phase peptide synthesis is exemplified in scheme B

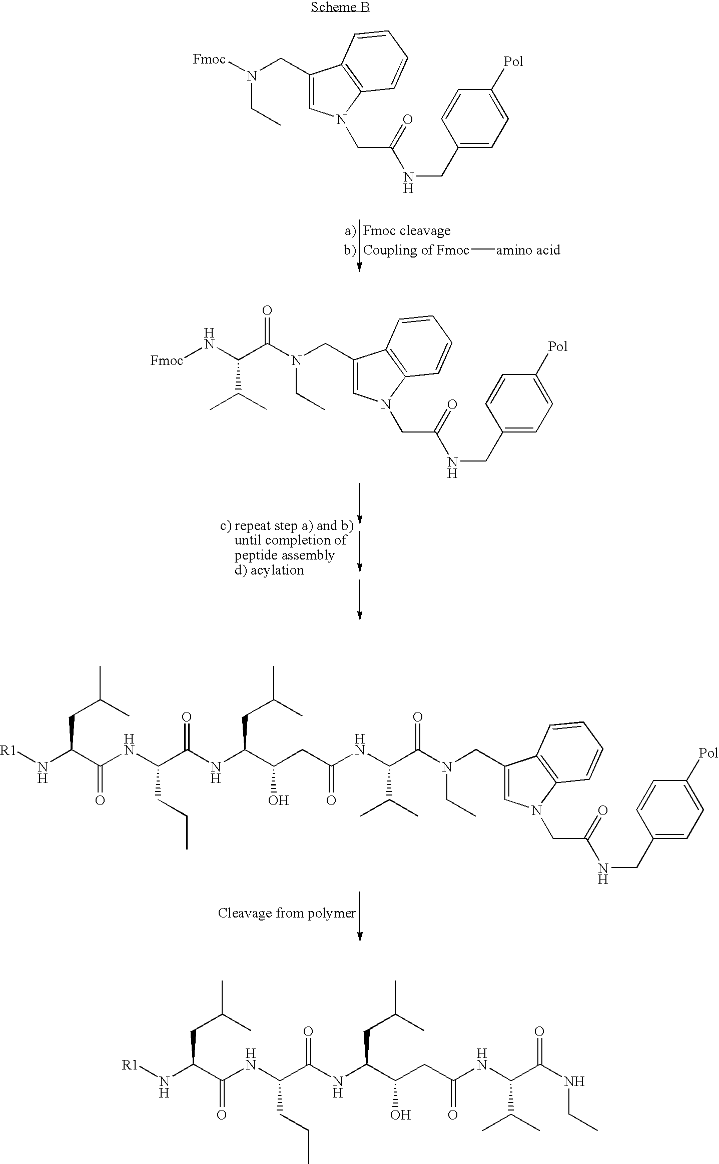

As a polymer commercially available [3-{[Ethyl-Fmoc-amino]-methyl}-indol-1-yl-acetyl AM resin (Indol resin, Novabiochem) is used. After cleavage of the Fmoc-group with piperidine in DMF (step a) the first amino acid is coupled with standard methods of peptide chemistry, e.g. HBTU/HOBt (step b). Step a and b are repeated until completion of the peptide chain an the terminal Fmoc-group is removed. The introduction of the N-terminal capping group can be achieved by standard acylation methods (step d). The C-terminal peptide N-ethlylamide is cleaved from the polymer by reaction with acids e.g. trifluoroacetic acid.

Scheme C illustrates the synthesis of peptides with modified C-termini. In this case the peptide is synthesized on a commercially available Fmoc-Val-TCP-resin. The stepwise elongation of the peptide chain (step a) is performed with standard methods. The last amino acid is coupled with a N-terminal Boc-protecting group. The cleavage from the polymer is possible with weak acids, e.g. hexafluoroisopropanol without cleavage of tBu-protecting groups (step b). The protected peptide acid is coupled with amines under standard amide coupling reactions, e.g. using N-(3-dimethylaminopropyl)-N-ethylcarbodiimide (step c). In the final reaction (step d) the tBu- and/or Boc-protecting groups are removed with trifluoroacetic acid.

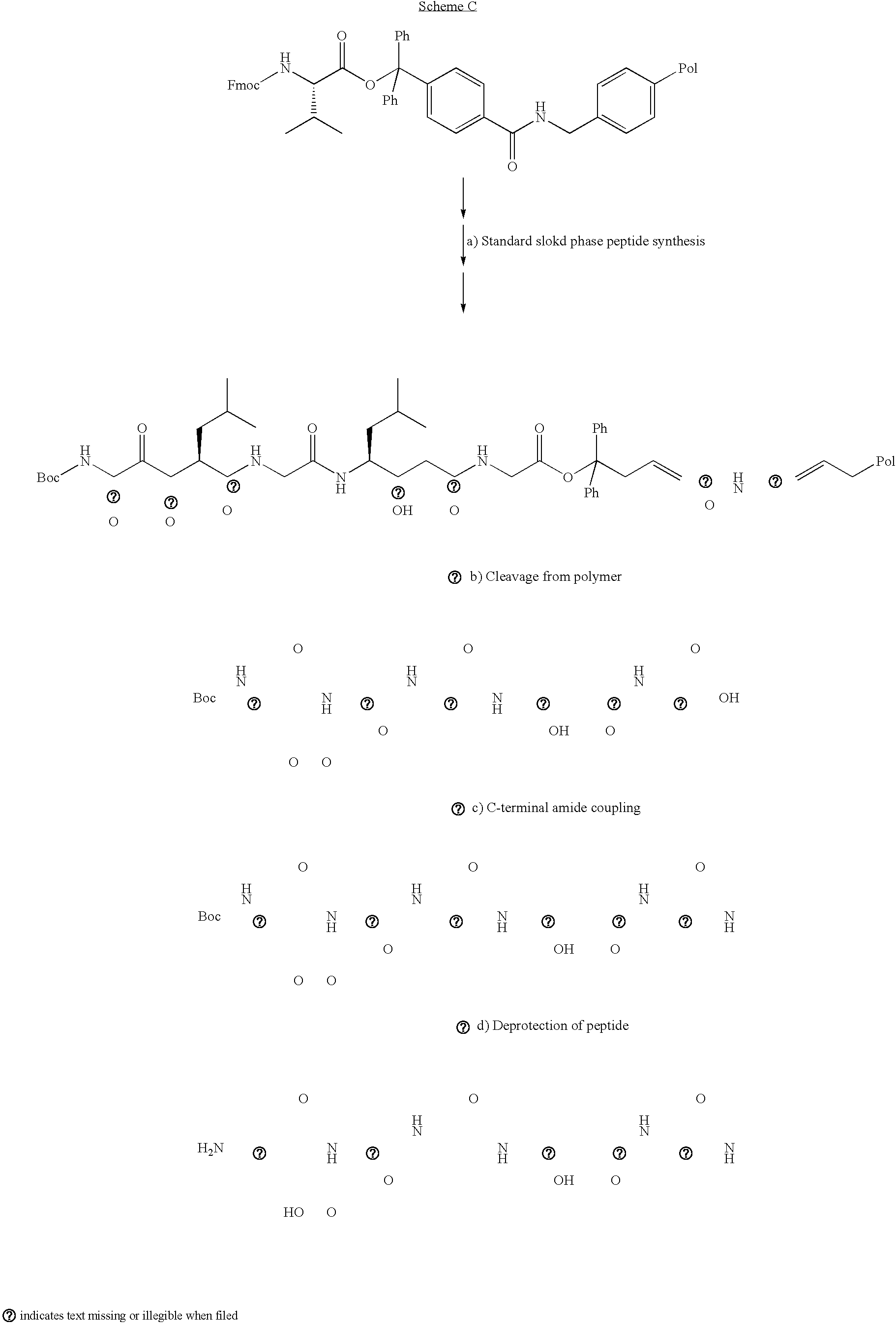

Part of the backbone of the compounds of the present invention is a statine moiety (Sta), -(3S,4S)-NH—CH (CH₂-i-propyl)-CH (OH)—(CH₂)—CO— which is commercially available 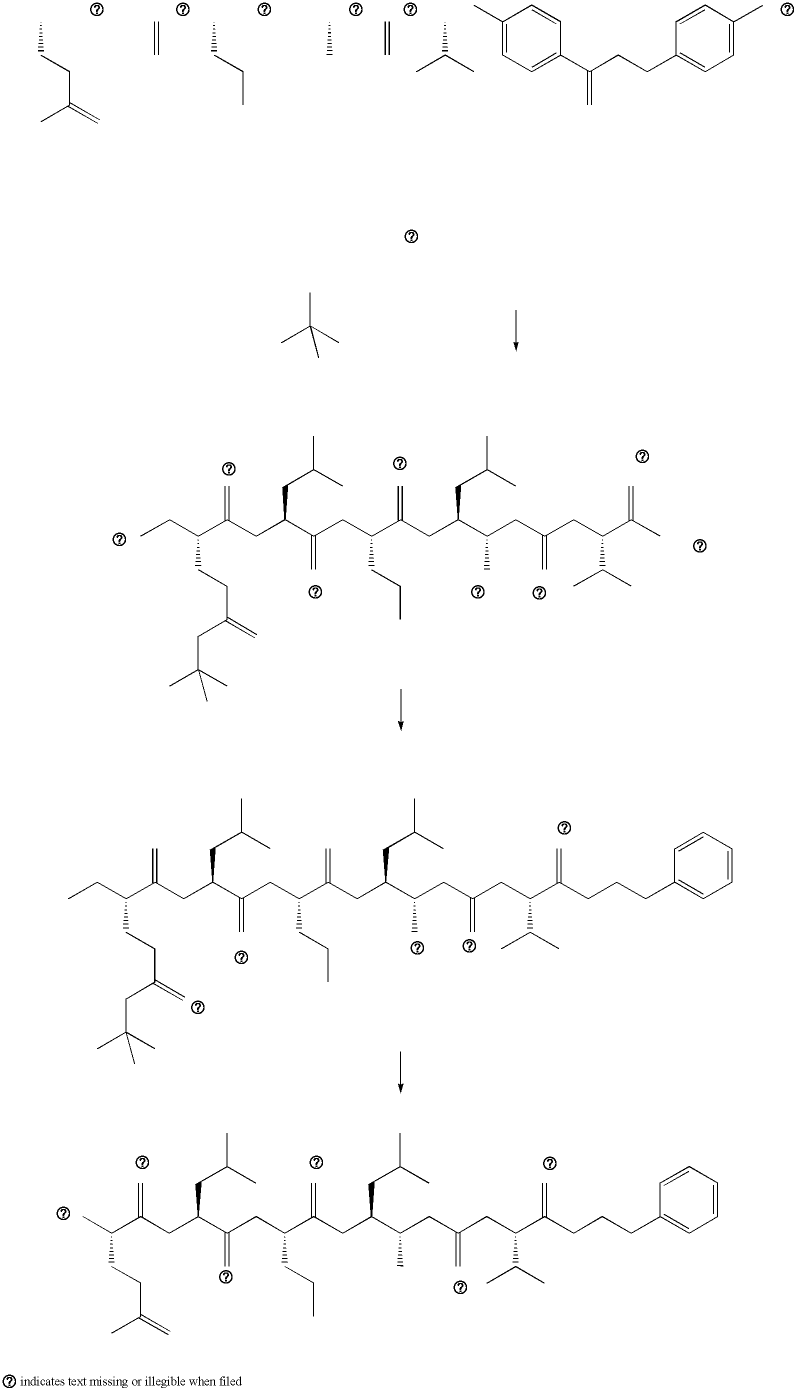
from various vendors. It can be readily prepared by methods disclosed in the literature and known to those skilled in the art.

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating patients with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i. e. single and recurrent lobal hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type Alzheimer's disease. The compounds and compositions of the invention are particularly useful for treating or preventing Alzheimer's disease. When treating or preventing these diseases, the compounds of the invention can either be used individually or in combination, as is best for the patient.

As used herein, the term “treatment” means that the compounds of the invention can be used in humans with at least a tentative diagnosis of disease. The compounds of the invention will delay or slow the progression of the disease thereby giving the individual a more useful life span.

The term “prevention” means that the compounds of the present invention are useful when administered to a patient who has not been diagnosed as possibly having the disease at the time of administration, but who would normally be expected to develop the disease or be at increased risk for the disease. The compounds of the invention will slow the development of disease symptoms, delay the onset of the disease, or prevent the individual from developing the disease at all.

Prevention also includes administration of the compounds of the invention to those individuals thought to be predisposed to the disease due to age, familial history, genetic or chromosomal abnormalities, and/or due to the presence of one or more biological markers for the disease, such as a known genetic mutation of APP or APP cleavage products in brain tissues or fluids.

The compounds of the invention are administered in a therapeutically effective amount. The therapeutically effective amount will vary depending on the particular compound used and the route of administration, as is known to those skilled in the art.

The compounds of the invention can be administered orally, parenterally, (IV, IM, depo-IM, SQ, and depo SQ), sublingually, intranasally, inhalative, intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration or aerosols for inhalative administration. Typically the compounds described above are formulated into pharmaceutical compositions using techniques and procedures well known in the art.

About 1 to 500 mg of a compound or mixture of compounds of the invention or a physiologically acceptable salt thereof is admixed with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action.

The compounds may be formulated as the sole pharmaceutically active ingredient in the composition or may be combined with one or more different active ingredients.

The concentration of the compound is effective for delivery of an amount upon administration that lessens or ameliorates at least one symptom of the disorder for which the compound is administered. Typically, the compositions are formulated for single dosage administration.

The compounds and compositions of the invention can be enclosed in multiple or single dose containers. The compounds and compositions according to the invention can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampules, vials and the like for parenteral administration; and patches, medipads, creams, and the like for topical administration, and optionally pre-filled inhalators for inhalative administration.

The concentration of active compound in the drug composition will depend on absorption, inactivation, and excretion rates of the active compound, the dosage schedule, and amount administered as well as other factors known to those of skill in the art.

It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, lozenges or troches.

Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Methods for preparation of such formulations are known to those skilled in the art.

The oral dosage forms are administered to the patient 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a patient may be started at one dose, that dose may be varied over time as the patient's condition changes.

The invention here is the new compounds of the invention and new methods of using the compounds of the invention. Given a particular compound of the invention and a desired dosage form, one skilled in the art would know how to prepare and administer the appropriate dosage form.

The compounds of the invention are used in the same manner, by the same routes of administration, using the same pharmaceutical dosage forms, and at the same dosing schedule as described above, for preventing disease or treating patients with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i. e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, and diffuse Lewy body type of Alzheimer's disease.

The compounds of the invention can be used in combination, with each other or with other therapeutic agents or approaches used to treat or prevent the conditions listed above. Such agents or approaches include: acetylcholine-esterase inhibitors such as tacrine (tetrahydroaminoacridine, marketed as COGNEXO), donepezil hydrochloride, (marketed as Aricept and rivastigmine; gamma-secretase inhibitors; anti-inflammatory agents such as cyclooxygenase II inhibitors; anti-oxidants such as Vitamin E and ginkolides; immunological approaches, such as, for example, immunization with A beta peptide or derivatives thereof or administration of anti-A beta peptide antibodies; neurotransmitter modulators like NS-2330; statins (HMG-CoA Reductase Inhibitors); and direct or indirect neurotropic agents such as Cerebrolysin (AIT-082) (Emilieu, 2000, Arch. Neurol. 57: 454), and other neurotropic agents of the future.

Most preferred are combinations with one or more additional active ingredient selected from the group consisting of atorvastatin, besipirdine, cevimeline, donepezil, eptastigmine, galantamine, glatiramer acetate, icopezil, ipidacrine, lazabemide, linopirdine, lubeluzole, memantine, metrifonate, milameline, nefiracetam, nimodipine, octreotide, rasagiline, rivastigmine, sabcomeline, sabeluzole, tacrine, valproate sodium, velnacrine, YM 796, Phenserine and zanapezil and/or with an antiinflammtory agents selected from the group consisting of rofecoxib, celecoxib, valdecoxib, nitroflurbiprofen, IQ-201, NCX-2216, CPI-1189, Colostrinin, ibuprofen, indomethacin, meloxicam and sulindac sulphide and/or one or more additional nerve growth factor and/or nerve growth modulator selected from the group consisting of: ABS-205, Inosine, KP-447, leteprinim, MCC-257, NS-521, NS-521, NS-2330, xaliproden.

It should be apparent to one skilled in the art that the exact dosage and frequency of administration will depend on the particular compounds of the invention administered, the particular condition being treated, the severity of the condition being treated, the age, weight, general physical condition of the particular patient, and other medication the individual may be taking as is well known to administering physicians who are skilled in this art.

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic and non-transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications W098/22597, WO00/03819, WO01/23533, and WO00/17369, as well as in literature publications (Hussain et. al., 1999, Mol. Cell. Neurosci. 14: 419-427; Vassar et. al., 1999, Science 286: 735-741; Yan et. al., 1999, Nature 402: 533-537; Sinha et. al., 1999, Nature 40: 537-540; and Lin et. al., 2000, PNAS USA 97: 1456-1460). Synthetic forms of the enzyme have also been described (W098/22597 and WO00/17369). Beta-secretase can be extracted and purified from human brain tissue and can be produced in cells, for example mammalian cells expressing recombinant enzyme.

Most preferably the assay is carried out as follows:

Assay Principle:

fluorescence quenching

Enzyme Source:

HEK293/APP cells stably expressing and secreting the ectodomain of BACE (aa 1-454) into the medium.

The cells are grown to confluency, washed with PBS and OptiMEM (Invitrogen) is added overnight. The medium containing BACE is collected and cell debris is removed by centrifugation.

The enzyme is stable for prolonged times (>3 mo) in OptiMEM at 4° C. or at −20° C.

Substrate:

The substrate peptide is obtained from Amersham Biotech and possesses a Cy3-fluorophore at the N-terminus and a Cy5Q-quencher at the C-terminus. The peptide sequence is: SEVNLDAEFK (derived from the APP sequence containing the Swedich mutation).

Assay Conditions:

The assay is performed in the presence of:
10 μl OptiMEM containing the ectodomain of BACE
100 μl water containing the desired concentration of compound with a max. conc. of
1% DMSO
1 μM substrate peptide
20 mM NaOAc, pH 4.4

total assay volume: 200 μl (adjusted with millipore water)

assay format: 96 well plate

incubation temperature: 30° C.

the cleavage of the substrate is recorded as kinetic for 30 min. at ex: 530 nm, em: 590 nm

the assay is started by the addition of substrate

Controls:

1.) no inhibitor present
2.) no enzyme present, instead OptiMEM conditioned from 293/APP cells is used
   IC₅₀ Determination:

For IC₅₀ determination different concentrations of compound were incubated in the assay. The relative compound inhibition potency is determined by calculating the concentration of compound that showed a 50% reduction in detected signal compared to the enzyme reaction signal in the control wells with no added compound.

Useful inhibitory compounds are effective to inhibit 50% of beta-secretase enzymatic activity at a concentration of less than 50 micromolar, preferably at a concentration of 10 micromolar or less, more preferably 1 micromolar or less, and most preferably 10 nanomolar or less.

The compounds of formula (I) exemplified below as examples 1 to 17 show IC₅₀ values of less than 10 micromolar.

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015” and 5,811,633, and in Games et. al., 1995, Nature 373: 523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Unless defined otherwise, all scientific and technical terms used herein have the same meaning as commonly understood by one of skill in the art to which this invention belongs. All patents and publications referred to herein are hereby incorporated by reference for all purposes. The definitions and explanations below are for the terms as used throughout this entire document including both the specification and the claims.

All temperatures are in degrees Celsius.
TLC refers to thin-layer chromatography. psi refers to pounds/in²,
THF refers to tetrahydrofuran,
DIEA refers to diisopropylethylamine,
DMF refers to dimethylformamide,
DCM refers to dichloromethane,
EDC refers to ethyl-1-(3-dimethylaminopropyl) carbodiimide or 1-(3-dimethylaminopropyl)-3-etliylcarbodiimide hydrochloride.
HOBt refers to 1-hydroxy benzotriazole hydrate,
HBTU refers to 2-(1H-Benzotriazole-1-yl)-1,1,3,3-tetramethyluronium hexafluorophosphate
NMM refers to N-methylmorpholine
NMP refers to N-methylpyrrolidone,
NBS refers to N-bromosuccinimide.
TEA refers to triethylamine.
BOC refers to 1,1-dimethylethoxy carbonyl or t-butoxycarbonyl,
CBZ refers to benzyloxycarbonyl,
FMOC refers to 9-fluorenylmethyl carbonate.
TFA refers to trifluoracetic acid,
CDI refers to 1,1′-carbonyldiimidazole.
tBu refers to tert.-butyl
Bzl refers to benzyl
Sta refers to (3S, 4S)-4-amino-3-hydroxy-6-methyl-heptanoic acid
Saline refers to an aqueous saturated sodium chloride solution.
Chromatography (column and flash chromatography) refers to purification/separation of compounds expressed as (support, eluent). It is understood that the appropriate fractions are pooled and concentrated to give the desired compound (s).
CMR refers to C-13 magnetic resonance spectroscopy, chemical shifts are reported in ppm (8) downfield from TMS.
NMR refers to nuclear (proton) magnetic resonance spectroscopy, chemical shifts are reported in ppm (d) downfield from TMS.
IR refers to infrared spectroscopy.
MS refers to mass spectrometry expressed as m/e, m/z or mass/charge unit
(M+H)⁺ refers to the positive ion of a parent plus a hydrogen atom.
EI refers to electron impact. CI refers to chemical ionization. FAB refers to fast atom bombardment.
HRMS refers to high resolution mass spectrometry.
Ether refers to diethyl ether, unless specified otherwise.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. When solvent pairs are used, the ratios of solvents used are volume/volume (v/v). When the solubility of a solid in a solvent is used the ratio of the solid to the solvent is weight/volume (wt/v).

BOP refers to benzotriazol-1-yloxy-tris (dimethylamino) phosphonium hexafluorophosphate.

### EXAMPLES

Without further elaboration, it is believed that one skilled in the art can, using the preceding description, practice the present invention to its fullest extent.

The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Synthesis of H-Glu-Ile-Nva-Sta-Val-Ala-OH (Example 2)

The peptide synthesis was performed on an Applied Biosystems peptide synthesizer ABI 433A using the pre-installed method FastMoc 0.10 Ω MonPrevPK.

Fmoc-Ala-Wang resin (Novabiochem, loading 0.74 mmol/g) (135.1 mg; 0.1 mmol) was added to the reaction vessel (8 ml) and DCM (3 ml) was added to swell the resin for 6 minutes under agitation. The DCM was removed and the resin was washed with NMP (four times; 2.5 ml). The deprotection of the Fmoc-group was performed by treatment of the resin with 22% piperidin/DMF for 2 and 7 minutes followed by washing the resin with NMP (12 times; 2.5 ml).

For the coupling of the amino acids NMP (2 ml), HBTU/HOBt in DMF (2 ml, 0.45 M, 0.9 mmol) and DIEA in DMF (1 ml; 2 M) were added to the amino acid cartridge Fmoc-Val-OH (339 mg; 1 mmol). The amino acid was dissolved by mixing for 6 minutes. This solution was added to the resin the reaction vessel was agitated for 2 hours. After completion of the coupling the reaction mixture was filtrated and resin was washed with NMP (12 times; 2.5 ml). The other amino acids Fmoc-Sta-OH, Fmoc-Nva-OH, Fmoc-Ile-OH and Fmoc-Glu(OtBu)-OH were incorporated in the same manner.

After completion of the peptide assembly the terminal Fmoc-group was deprotected as described above. The resin was transferred into a 10 ml syringe equipped with a filter and washed with DCM (5 times; 4 ml) by hand. The resin was treated with a solution of 95% TFA/water (5 ml). After 30 minutes the solution was filtrated and the resin was washed with DCM (2 times, 3 ml). The combined solutions were evaporated under reduced pressure and the resulting oil was treated with diethyl ether to precipitate the peptide. The crude peptide was purified by preparative reversed phase HPLC applying an acetonitrile/water gradient. The product gave satisfactory analytical data. HPLC>99%; ES-MS: m/z=687.4 ([M+H]+)

The examples 3-6 of Table 1 were synthesized analogously.

### Synthesis of Glutaryl-Ile-Nva-Sta-Val-NHEt (Example 13)

The peptide synthesis was performed on an Applied Biosystems peptide synthesizer ABI 433A using the pre-installed method FastMoc 0.25Ω MonPrevPK.

3-((Ethyl-fmoc-amino)-methyl)-1-indol-1yl-acetyl AM resin (Novabiochem, loading 0.87 mmol/g) (287.4 mg; 0.25 mmol) was added to the reaction vessel (41 ml) and DCM (5 ml) was added to swell the resin for 6 minutes under agitation. The DCM was removed and the resin was washed with NMP (five times; 5 ml). The deprotection of the Fmoc-group was performed by treatment of the resin with 22% piperidin/DMF for 2 and 7 minutes followed by washing the resin with NMP (12 times; 5 ml).

For the coupling of the amino acids NMP (2 ml), HBTU/HOBt in DMF (2 ml, 0.45 M, 0.9 mmol) and DIEA in DMF (1 ml; 2 M) were added to the amino acid cartridge Fmoc-Val-OH (339 mg; 1 mmol). The amino acid was dissolved by mixing for 6 minutes. This solution was added to the resin and the reaction vessel was agitated for 2 hours. After completion of the coupling the reaction mixture was filtrated and resin was washed with NMP (12 times; 5 ml). The other amino acids Fmoc-Sta-OH, Fmoc-Nva-OH, Fmoc-Ile-OH were incorporated in the same manner.

After completion of the peptide assembly the terminal Fmoc-group was deprotected as described above. The resin was transferred into a 10 ml syringe equipped with a filter and a solution of glutaric anhydride (114.1 mg; 0.1 mmol), DIEA (513.7 μl; 3 mmol) and DMF (3 ml) was added. The suspension was agitated for two hours. The resin was washed with DMF (5 times; 5 ml) and DCM (5 times; 5 ml) by hand. The resin was treated with a solution of 95% TFA/water (5 ml). After 30 minutes the solution was filtrated and the resin was washed with DCM (2 times, 3 ml). The combined solutions were evaporated under reduced pressure and the resulting oil was treated with diethyl ether to precipitate the peptide. The crude peptide was purified by preparative reversed phase HPLC applying an acetonitrile/water gradient. The product gave satisfactory analytical data. HPLC>99%; ES-MS: m/z=628.4 ([M+H]+)

The examples 7-27 of Table 1 were synthesized analogously.

### Synthesis of H-Glu-Leu-Nva-Sta-Val phenethylamide (Example 1)

1) Synthesis of N-α-Boc-glutamyl-γ-tBu-ester-leucyl-norvalyl-statyl-valine

The synthetic pentapeptide N-α-Boc-L-glutamyl-γ-tBu-ester-leucyl-norvalyl-statyl-valine was prepared by solid phase peptide synthesis using Fmoc/tBu-chemistry and Fmoc-valine-diphenylmethylbenzoyl-amidomethyl-polystyrene resin (Fmoc-Val-TCP-resin) as starting material.

1a) Synthesis of Fmoc-statyl-Val-TCP-resin

Fmoc-Val-TCP-resin (commercially available from PepChem Goldammer&Clausen), capacity 0.78 mmol/g (90 mg, 70.2 μmol) was washed twice with DMF 82 ml) and deprotected by shaking with 30% piperidine/DMF (1 ml) at room temperature for 15 min. The resin was filtered off and was washed with DMF, dichloromethane, methanol and dichloromethane (3 times each, 1.2 ml each). The resin was incubated (15 min) with dry THF (1 ml) and DIEA (1 ml) and filtered off.

Fmoc-Statine (83.7 mg, 210.6 μmol) was dissolved in a solution of bis(trichloro-methyl)carbonate (68 mM) in dry THF (3.1 ml). Sym.-collidine was added (834 μl, 630 μmol). After incubation (1 min) the resulting suspension was added to the resin and the mixture was shaken at room temperature for 16 h. The resin was filtered off and was washed with THF, DMF and dichloromethane (3 times each, 1.2 ml each).

1b) Synthesis of norvalyl-statyl-Val-TCP-resin

Fmoc-statyl-Val-TCP-resin (70.2 μmol) was deprotected by shaking with 30% piperidine/DMF (1 ml) at room temperature for 15 min. The resin was filtered off and was washed with DMF, dichloromethane, methanol and dichloromethane (3 times each, 1.2 ml each). The resin was incubated (15 min) with dry THF (1 ml) and DIEA (1 ml) and filtered off. Fmoc-norvaline (71.4 mg, 210.6 μmol) was dissolved in a solution of bis(trichloro-methyl)carbonate (68 mM) in dry THF (3.1 ml). Sym.-collidine was added (834 μl, 630 μmol). After incubation (1 min) the resulting suspension was added to the resin and the mixture was shaken at room temperature for 4 h. The resin was filtered off and was washed with THF, DMF and dichloromethane (3 times each, 1.2 ml each).

1c) Synthesis of Fmoc-leucyl-norvalyl-statyl-Val-TCP-resin

Fmoc-norvalyl-statyl-Val-TCP-resin (70.2 μmol) was deprotected by shaking with 30% piperidine/DMF (2 ml) at room temperature for 15 min. The resin was filtered off and was washed with DMF, dichloromethane, methanol and dichloromethane (3 times each, 1.2 ml each). Fmoc-leucine (173.7 mg, 491.4 μmol) was dissolved in a solution of N-hydroxybenzotriazole (0.5 M) in DMF (0.98 ml). N,N′-Diisopropylcarbodiimide was added (77.4 μl, 500 μmol) and the mixture was shaken at room temperature for 50 min. The resin was filtered off and was washed with DMF (9 times, 1.2 ml each).

1d) Synthesis of N-α-Boc-glutamyl-γ-tBu-ester-leucyl-norvalyl-statyl-Val-TCP-resin Fmoc-leucyl-norvalyl-statyl-Val-TCP-resin (70.2 μmol) was deprotected by shaking with 30% piperidine/DMF (1 ml) at room temperature for 15 min. The resin was filtered off and was washed with DMF, dichloromethane, methanol and dichloromethane (3 times each, 1,2 ml each). N-α-t.Boc-glutamic acid-γ-t.butyl ester (149.1 mg, 491,4 μmol) was dissolved in a solution of N-hydroxybenzotriazole (0.5 M) in DMF (0.98 ml). N,N′-Diisopropylcarbodiimide was added (77.4 μl, 500 μmol) and the mixture was shaken at room temperature for 50 min. The resin was filtered off and was washed with DMF and dichloromethane (4 times each, 1.2 ml each).

1e) Synthesis of N-α-Boc-glutamyl-γ-tBu-ester-leucyl-norvalyl-statyl-valine N-α-Boc-glutamyl γ-tBu-ester-leucyl-norvalyl-statyl-Val-TCP-resin (70.2 μmol) was treated two times with a solution of hexafluoroisopropanol in dichloromethane (1:1, v/v, 2 ml) for 30 min and filtered off the resin. The cleavage solutions were pooled and the solvents were evaporated and the residue was dissolved in t.butyl alcohol/water (4:1, v/v, 5 ml) by sonication and lyophilised.

Yield: 48 mg, colourless powder.

2) Synthesis of glutamyl-leucyl-norvalyl-statyl-valine phenethylamide

2a) Synthesis of N-α-Boc-glutamyl γ-tBu-ester-leucyl-norvalyl-statyl-valine phenethylamide

A solution of N-α-glutamyl γ-tBu-ester-leucyl-norvalyl-statyl-valine (45 mg, 51 μmol) and N-(3-dimethylaminopropyl)-N-ethylcarbodiimide hydochloride (9.8 mg, 51 μmol) was dissolved in THF (2 ml) and stirred at room temperature for 1 h. 2-Phenethylamine (9.6 μl, 76.5 μmol) was added and the mixture was shaken at room temperature for 14 h. The solvent was removed in vacuo and the residual was dissolved in dichloromethane (10 ml) and extracted with 5% NaHCO3, 5% acetic acid and water (each 3×10 ml). After drying over sodium sulfate the solvent was evaporated.

Yield: 49.8 mg, colourless powder.

2b) Synthesis of H-Glu-Leu-Nva-Sta-Val phenethylamide

The residue 2a was treated with trifluoroacetic acid containing 5% of triisopropylsilane and 2.5% of water for 3 h. Trifluoroacetic acid was removed in vacuo and the residue was dissolved in tert.butyl alcohol/water 4:1 and lyophilized.

Yield: 38.8 mg (77% related to resin capacity), colourless powder.

3) Electrospray Mass Spectrometry

The peptide was dissolved in tert.butyl alcohol/water 4:1 (1 mg/ml). The solution was diluted 1:10 with acetonitrile/water 1:1 containing 0. 1% formic acid. For electrospray mass spectrometry, a triple-quadrupol mass spectrometer VG quattro II was employed, equipped with an nebulizer-assisted electrospray source. 10 μl of the solutions were measured by using a Gilson XL 232 autosampler (Abimed).

Calcd.: 718.4

Found 719.4 [M+H]+

4) HPLC Purification

Crude product (38.8 mg, 12.9 mg/separation, dissolved in 3 ml methanol/water=1:1, v/v) was purified by preparative HPLC:
Column: Thermo-Hypersil-Keystone RP-18, 5 μm, 100×21.2 mm, 30 ml/min
Mobile phase:
Eluent A: Water/0.1% TFA (v/v),
Eluent B: Acetonitril/0.1% TFA (v/v)
Gradient: 60% A to 40% B within 5 min; 40%B to 100% B within 19 min.
Fractions containing the product (>95%) were identified by HPLC-MS

Yield after purification: 10 mg

TABLE I

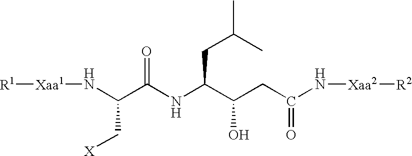

| | | | | | | ES-MS |
|---|---|---|---|---|---|---|
| Example No | R¹ | Xaa¹ | X | Xaa² | R² | (M + H)⁺ |
| 1 | H | -Glu-Leu- | —CH₂—CH₃ | -Val-Ala- | —CH₂-Bzl | 719.4 |
| 2 | H | -Glu-Leu- | —CH₂—CH₃ | -Val-Ala- | —OH | 687.4 |
| 3 | H | -Glu-Leu- | —CH₂—CH₃ | -Val-Ala- | —OH | 687.4 |
| 4 | H | -Glu-Leu- | —S—CH₃ | -Val-Ala- | —OH | 705.8 |
| 5 | H | -Glu-Leu- | —CH₂—S—CH₃ | -Val-Ala- | —OH | 719.9 |
| 6 | H | -Glu-Leu- | -cyclopropyl | -Val-Ala- | —OH | 699.8 |
| 7 | Pyroglutaminoyl | -Leu- | —CH₂—CH₃ | -Val- | —NH—CH₂—CH₃ | 625.8 |
| 8 | 4-(MeCO—NH)—Ph—CO— | -Leu- | —CH₂—CH₃ | -Val- | —NH—CH₂—CH₃ | 675.9 |
| 9 | 3-(MeCO—NH)—Ph—CO— | -Leu- | —CH₂—CH₃ | -Val- | —NH—CH₂—CH₃ | 675.9 |
| 10 | Ac—NH—CH₂—CH₂—CO— | -Leu- | —CH₂—CH₃ | -Val- | —NH—CH₂—CH₃ | 627.8 |
| 11 | Ac—NH—(CH₂)₃—CO— | -Leu- | —CH₂—CH₃ | -Val- | —NH—CH₂—CH₃ | 641.8 |
| 12 | HOCO—(CH₂)₃—CO— | -Leu- | —CH₂—CH₃ | -Val- | —NH—CH₂—CH₃ | 628.8 |
| 13 | HOCO—(CH₂)₃—CO— | -Ile- | —CH₂—CH₃ | -Val- | —NH—CH₂—CH₃ | 628.7 |
| 14 | HOCO—(CH₂)₃—CO— | -Leu- | -cyclopropyl | -Val- | —NH—CH₂—CH₃ | 640.8 |
| 15 | 4-(HOCO)—Ph—CO— | -Leu- | —CH₂—CH₃ | -Val- | —NH—CH₂—CH₃ | 662.8 |
| 16 | Ac-Nip- | -Leu- | —CH₂—CH₃ | -Val- | —NH—CH₂—CH₃ | 661.9 |
| 17 | Bz-Nip- | -Leu- | —CH₂—CH₃ | -Val- | —NH—CH₂—CH₃ | 730.0 |
| 18 | 4-(OH)—Ph—CO— | -Leu- | —CH₂—CH₃ | -Val- | —NH—CH₂—CH₃ | 634.8 |
| 19 | 3-(OH)—Ph—CO— | -Leu- | —CH₂—CH₃ | -Val- | —NH—CH₂—CH₃ | 634.8 |
| 20 | HOCO—(CH₂)₃—CO— | -Ile- | —CH₂—CH₃ | -Phe- | —NH—CH₂—CH₃ | 676 |
| 21 | HOCO—(CH₂)₃—CO— | -Ile- | —CH₂—CH₃ | -Chg- | —NH—CH₂—CH₃ | 669 |
| 22 | HOCO—(CH₂)₃—CO— | -Ile- | —CH₂—CH₃ | -Abu- | —NH—CH₂—CH₃ | 615 |
| 23 | HOCO—(CH₂)₃—CO— | -Ile- | —CH₂—CH₃ | -Cpa- | —NH—CH₂—CH₃ | 640 |
| 24 | HOCO—(CH₂)₃—CO— | -Ile- | —CH₂—CH₃ | -Val- | —NH—CH₂—CH₃ | 629 |
| 25 | HOCO—(CH₂)₃—CO— | -Ile- | —CH₂—CH₃ | -Nva- | —NH—CH₂—CH₃ | 629 |
| 26 | 3-(MeO)—Ph—CO— | -Leu- | —CH₂—CH₃ | -Val- | —NH—CH₂—CH₃ | 648.7 |
| 27 | 3-(HOCO)—Ph—CO— | -Leu- | —CH₂—CH₃ | -Val- | —NH—CH₂—CH₃ | 662.8 |

### EXAMPLE 28

### Examples of Pharmaceutical Formulations

| A) Tablets | per tablet |
|---|---|
| active substance (Example 1) | 50 mg |
| lactose | 170 mg |
| corn starch | 260 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet |
|---|---|
| active substance (Example 1) | 40 mg |
| corn starch | 210 mg |
| lactose | 65 mg |
| microcrystalline cellulose | 40 mg |
| polyvinylpyrrolidone | 20 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) Coated tablets | per coated tablet |
|---|---|
| Active substance (Example 1) | 5 mg |
| Corn starch | 41.5 mg |
| Lactose | 30 mg |
| Polyvinylpyrrolidone | 3 mg |
| Magnesium stearate | 0.5 mg |
| | 80 mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| D) Capsules | per capsule |
|---|---|
| Active substance (Example 1) | 25 mg |
| Corn starch | 283.5 mg |
| Magnesium stearate | 1.5 mg |
| | 310 mg |

The substance and corn starch are mixed and moistened with water. The moist mass is screened and dried. The dry granules are screened and mixed with magnesium stearate. The finished mixture is packed into size 1 hard gelatine capsules.

| E) Ampoule solution | | |
|---|---|---|
| active substance (Example 1) | 0.5 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 0.5 mg, 2.5 mg and 5.0 mg of active substance.

| F) Suppositories | |
|---|---|
| Active substance (Example 2) | 30 mg |
| Solid fat | 1670 mg |
| | 1700 mg |

The solid fat is melted. The ground active substance is homogeneously dispersed at 40° C. It is cooled to 38° C. and poured into slightly chilled suppository moulds.

1. A compound of the formula 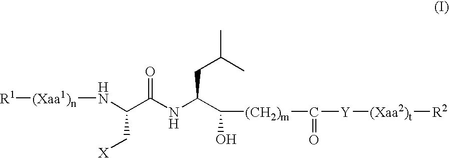
wherein
R¹ represents a hydrogen atom or a group selected from the formulae (A) and (B)
(A) R³—CO—(CH₂)ₛ—CO—,
in which
R³ represents R⁴-Z¹ with Z¹ being O or NR⁵, R⁴, R⁵ being each independently hydrogen or C₁₋₆ alkyl, and
s is an integer from 1 to 4;
(B) R⁶—CO—
in which
R⁶ represents a C₁₋₆alkyl group, a C₁₋₆ haloalkyl group or a phenyl group being optionally substituted by one or more substituents selected from the group consisting of halogen, C₁₋₆ alkyl, C₁₋₆ alkoxy, C₁₋₆ haloalkyl, C₁₋₆ haloalkoxy, amino, C₁₋₆ alkylamino, di-(C₁₋₆ alkyl)-amino, C₁₋₆ alkoxycarbonyl, formyl, carboxy, hydroxy, cyano, SO₃H and nitro;
Xaa¹ each independently represent an amino acid or the N-alkylated derivative thereof, at least one of which being N-terminally linked to R¹;
n is 0 or an integer from 1 to 3;
Y represents a single bond, or if t is 0, a spacer group selected from —O— and —NH—;
R² represents a hydroxy group or a group of formula (C)
(C) -Z²-R⁷
in which
Z represents O or NR⁸,
R⁷ represents
(a) a C₁₋₆ alkyl group being optionally substituted by one or more substituents selected from the group consisting of halogen, C₃₋₈-cycloalkyl, phenyl, C₁₋₆ alkoxy, C₁₋₆ haloalkoxy, amino, C₁₋₆ alkylamino, di-(C₁₋₆ alkyl)-amino, C₁₋₆ alkoxycarbonyl, formyl, carboxy, hydroxy, cyano and nitro, or
(b) a phenyl group being optionally substituted by one or more substituents selected from the group consisting of halogen, C₁₋₆ alkyl, C₁₋₆ alkoxy, C₁₋₆ haloalkyl, C₁₋₆ haloalkoxy, amino, C₁₋₆ alkylamino, di-(C₁₋₆ alkyl)-amino, C₁₋₆ alkanoylamino, C₁₋₆ alkoxycarbonyl, formyl, carboxy, hydroxy, cyano and nitro,
R² represents a hydrogen atom or C₁₋₆ alkyl group;
Xaa² each independently represent an amino acid or the N-alkylated derivative thereof, in which the amino group of the N-terminally amino acid may have been replaced by Y, and one of which being C-terminally linked to R²;
t is 0 or an integer from 1 to 3;
X is selected from ethyl, thiomethyl and C₃-C₈-cycloalkyl; and
m is 1 or 2,
or a pharmaceutically acceptable salt or solvate thereof.

2. A compound according to claim 1, wherein
Xaa¹ each independently is selected from the group of amino acids consisting of: Leu, Ile, Nva, Abu, Glu, Tie, Phg, Val, allo-Ile, Cpa, Met, Thr, Chg, S-Methylcystein, D-Leu, Nip, CBA (Cyanobutyric acid) and Allyl-Glycin; and
n is 1 or 2.

3. A compound according to claim 1, wherein
Xaa² each independently is selected from the group of amino acids consisting of: Val, Ala, Leu, Ile, Nva, Abu, Cha, Tle, Phg, Glu, Nle, Phe, His, Ser, Cpa, and Asp; and s is 1 or 2.

4. A compound according to claim 2, wherein
Xaa² each independently is selected from the group of amino acids consisting of: Val, Ala, Leu, Ile, Nva, Abu, Cha, Tle, Phg, Glu, Nle, Phe, His, Ser, Cpa, and Asp; and
s is 1or 2.

5. A compound according to claim 1, wherein
m represents 1.

6. A compound selected from the formulae (IA) through (ID): 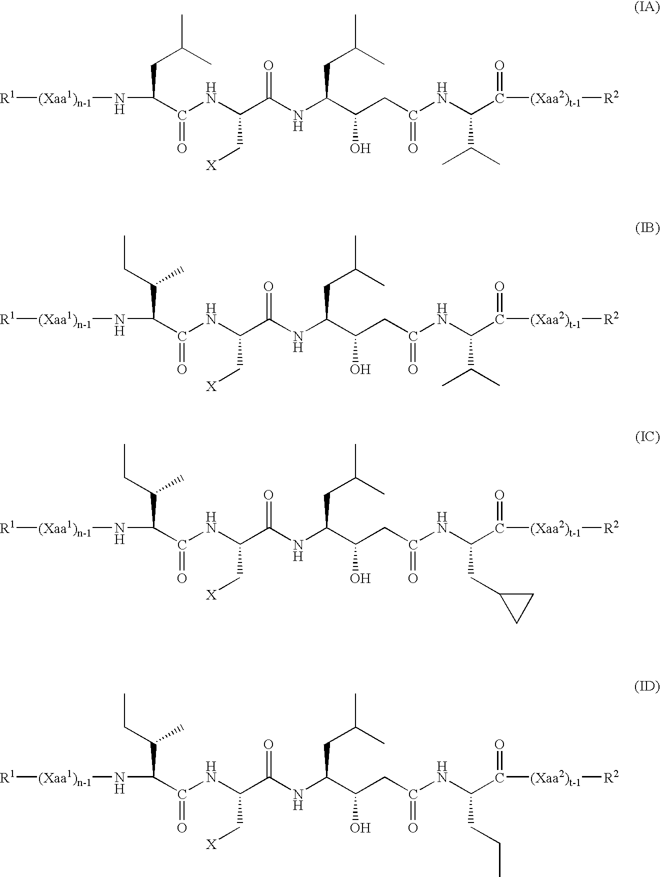
in which R¹ , R², Xaa¹, Xaa², n and t are as defined in claim 1, and X represents ethyl, thiomethyl or cyclopropyl; or a pharmaceutically acceptable salt or solvate thereof.

7. A pharmaceutical composition comprising a compound according to claim 1 or a pharmaceutically acceptable salt or solvate thereof, and a pharmaceutically acceptable carrier or diluent.

8. A pharmaceutical composition comprising a compound according to claim 6 or a pharmaceutically acceptable salt or solvate thereof, and a pharmaceutically acceptable carrier or diluent.

9. A pharmaceutical composition according to claim 7, which further comprises an active ingredient selected from the group consisting of: atorvastatin, besipirdine, cevimeline, donepezil, eptastigmine, galantamine, glatiramer acetate, icopezil, ipidacrine, lazabemide, linopirdine, lubeluzole, memantine, metrifonate, milameline, nefiracetam, nimodipine, octreotide, rasagiline, rivastigmine, sabcomeline, sabeluzole, tacrine, valproate sodium, velnacrine, YM 796, Phenserine and zanapezil.

10. A pharmaceutical composition according to claim 7, which further comprises an antiinflammtory agent selected from the group consisting of: rofecoxib, celecoxib, valdecoxib, nitroflurbiprofen, IQ-201, NCX-2216, CPI-1189, Colostrinin, ibuprofen, indomethacin, meloxicam, sulindac sulphide.

11. A pharmaceutical composition according to claim 9, which further comprises an antiinflammtory agent selected from the group consisting of: rofecoxib, celecoxib, valdecoxib, nitroflurbiprofen, IQ-201, NCX-2216, CPI-1189, Colostrinin, ibuprofen, indomethacin, meloxicam, sulindac sulphide.

12. A pharmaceutical composition according to claim 7, which further comprises a nerve growth factor or a nerve growth modulator selected from the group consisting of: ABS-205, Inosine, KP447, leteprinim, MCC-257, NS-521, and xaliproden.

13. A pharmaceutical composition according to claim 9, which further comprises a nerve growth factor or a nerve growth modulator selected from the group consisting of: ABS-205, Inosine, KP447, leteprinim, MCC-257, NS-521, and xaliproden.

14. A pharmaceutical composition according to claim 11, which further comprises a nerve growth factor or nerve growth modulator selected from the group consisting of: ABS-205, Inosine, KP-447, leteprinim, MCC-257, NS-521, and xaliproden.

15. A method of treating or preventing a disease or condition in a patient, comprising administering the compound according to claim 1, wherein the disease or condition is selected from the group consisiting of: Alzheimer's disease, Down's syndrome, MCI (“Mild Cognitive Impairment”), Hereditary Cerebral Hemorrhage with Amyloidosis of the Dutch-Type, Cerebral Amyloid Angiopathy, Traumatic Brain injury, Stroke, Dementia, Parkinson's Disease and Parkinson's Syndrome, and central or peripheral amyloid diseases.

16. A method of treating or preventing a disease or condition in a patient, comprising administering the pharmaceutical composition according to claim 7, wherein the disease or condition is selected from the group consisiting of: Alzheimer's disease, Down's syndrome, MCI (“Mild Cognitive Impairment”), Heriditary Cerebral Hemorrhage with Amyloidosis of the Dutch-Type, Cerebral Amyloid Angiopathy, Traumatic Brain injury, Stroke, Dementia, Parkinson's Disease and Parkinson's Syndrome, and central or peripheral amyloid diseases.

17. A method for inhibiting β-secretase activity, comprising exposing said β-secretase to an effective inhibitory amount of a compound of claim 1.

18. A method for inhibiting β-secretase activity, comprising exposing said β-secretase to an effective inhibitory amount of a compound of formula IA of claim 6.
